(19) United States
(12) Patent Application Publication
(10) Pub. No.: US 2023/0363935 A1
(43) Pub. Date: Nov. 16, 2023

(54) SPLINT DEVICE AND SPLINT SYSTEM COMPRISING SAME

(71) Applicant: X-CEN-TEK GmbH & Co. KG, Wardenburg (DE)

(72) Inventor: Andreas HARMS, Bad Zwischenahn (DE)

(73) Assignee: X-CEN-TEK GmbH & Co. KG, Wardenburg (DE)

(21) Appl. No.: 18/315,818

(22) Filed: May 11, 2023

(30) Foreign Application Priority Data
May 13, 2022 (DE) 202022102644.0

(51) Int. Cl. A61F 5/058

(52) U.S. Cl. CPC A61F 5/05825; A61F 5/05841

(57) ABSTRACT

A splint device includes a thin-walled strip as a stiffening component. A first fiber material is disposed on one side of the thin-walled strip. The splint device also includes a strap of a second fiber material. The one and the other fiber material function in combination as a hook and loop fastener.

### DESCRIPTION

The invention relates to a splint device in accordance with the preamble of claim 1. Moreover, the invention relates to a splint system comprising such a splint device.

Traction splints first known as so-called splints are most commonly used for treating severe fractures of the center of the femur, but also for treating fractures of other limbs. The femur is the longest and strongest bone of the human body. The muscles surrounding it are also very strong. When the femur fractures, the surrounding muscles may spasm, causing the bone ends to be pulled past each other, which causes immense pain, internal or external bleeding, and muscle and nerve damage.

A traction splint counteracts this by splinting the broken limb while applying traction to the fracture.

For this purpose, the traction splint comprises a rod, which is placed parallel to the broken leg and is supported against the pelvis, and acts as a splint on the femur by wrapping bandaging material around it. A traction device is disposed on the distal end of the rod which pulls the foot of the broken leg away from the pelvis.

In case of bone fractures in less muscular limbs, the traction splint is oversized. In these cases, the application of a tractive force is also not entirely necessary. Furthermore, the pack size of traction splints is disadvantageous for carrying them for rendering emergency first aid at the scene of an accident. Another disadvantage is the complex, time-consuming application of the traction splint.

Also known is a splint device for immobilizing broken bones and other injuries. The splint device comprises a core made of thin-walled aluminum covered with a coating, for example made of a foam material, such as polyethylene foam. The splint device may be stored and carried compactly rolled up to a roll. Unrolled, it initially appears as a rectangular, flat strip. A limb may easily be splinted with the splint device by deforming the strip along the limb to form a groove having a U-shaped cross-section stiffening the core made of aluminum sheet and fixing it to the limb with bandages.

A disadvantage of the splint device is its application, which necessitates the use of additional objects and bandaging materials that need to be carried along.

It is an object of the invention to provide a splint device that may be carried along in compact fashion and universally applied in a simple and quick manner. Moreover, it is an object of the invention to develop a splint system comprising such a splint device with its advantages.

The object is achieved by the features of the independent claim. Advantageous embodiments are given in the claims, the drawings, and the following description, including the description of the drawings.

A first subject of the invention thus relates to a splint device which can be used as a splinting means for fractures and other injuries. The splint device may also be referred to as a splint. It comprises a thin-walled strip as a stiffening component. For this purpose, an aluminum sheet is suitable, for example. Alternatively or additionally, a thin polymer sheet may form all or part of the stiffening component.

A first fiber material is disposed on at least one side of the thin-walled strip. The side of the splint provided with the first fiber material is also referred to as the loop side.

In addition, the splint device comprises at least one strap of a second fiber material.

One of the fiber materials comprises flexible hooks, the other one comprises loops.

In combination, the one and the other fiber material function as a hook and loop fastener.

A second subject of the invention relates to a system of a splint device described above that may be used as a splinting means for fractures and other injuries and of an additional hook strap comprising the second fiber material and fixing the splint device to a patient by means of the mutual interaction of the loop side and the hook strip.

The splint device may have individual or a combination of the features previously and/or subsequently described in connection with the system, just as the system may have and/or implement individual or a combination of multiple features previously and/or subsequently described in connection with the splint device.

The splint device and/or the system may alternatively or additionally have individual or a combination of several features described in the introduction in connection with the prior art and/or described in the following description of the embodiments shown in the drawings.

Additional advantages compared to the prior art that go beyond the complete achievement of the object and/or the advantages mentioned above for the individual features are described below.

The invention will be discussed in more detail with the help of the example embodiments illustrated in the drawings. The proportions of the individual elements to one another in the figures do not always correspond to the real proportions, since some shapes are shown in simplified form and other shapes are shown enlarged in relation to other elements for better illustration. Identical reference symbols are used for elements of the invention that are the same or have the same effect. Furthermore, for the sake of clarity, only reference symbols are shown in the individual figures that are necessary for the description of the respective figure. The illustrated embodiments only represent examples of how the invention can be configured and do not represent any final limitation. The figures illustrate the following schematically:

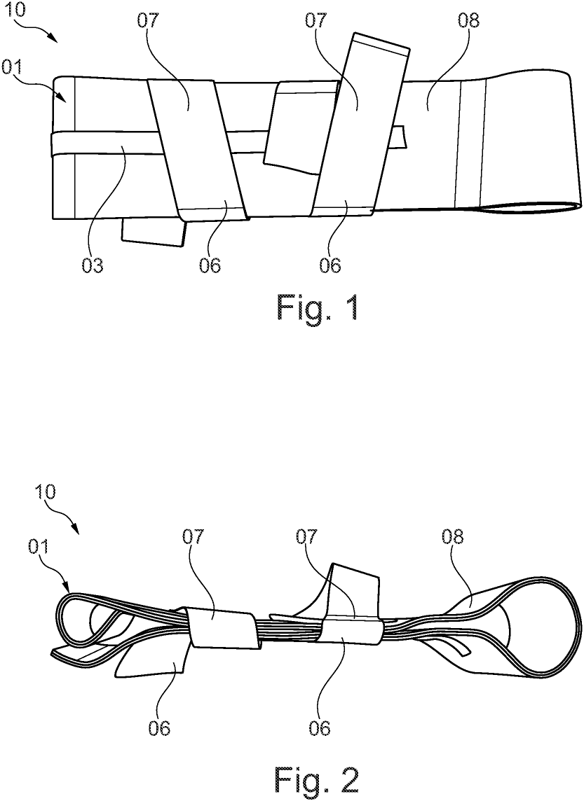

FIG. 1 depicts a plan view of an initial state of a splint device 01 and a system 10 comprising said device.

FIG. 2 depicts a side view of the splint device 01 and the system 10 of FIG. 1 comprising said device.

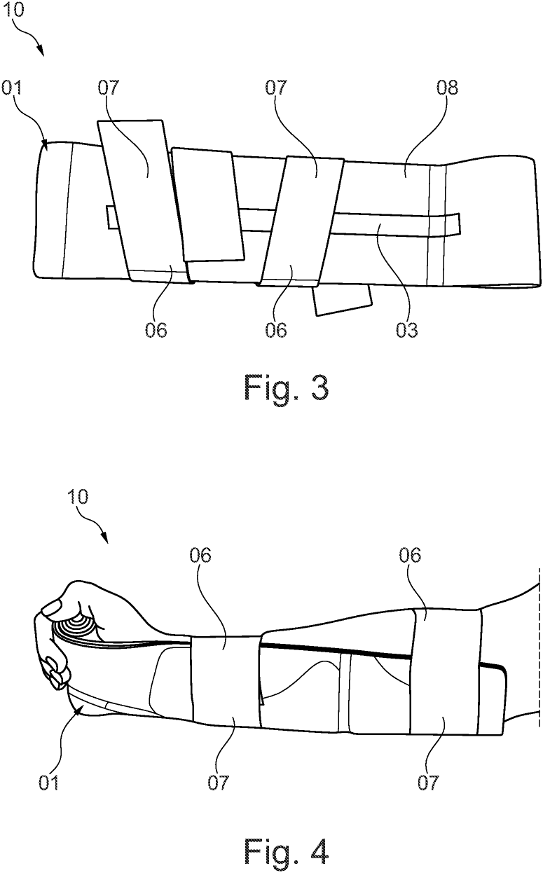

FIG. 3 depicts a bottom view of the splint device 01 and the system 10 of FIG. 1 comprising said device.

FIG. 4 depicts a perspective view of an application state of the splint device 01 and a system 10 comprising said device.

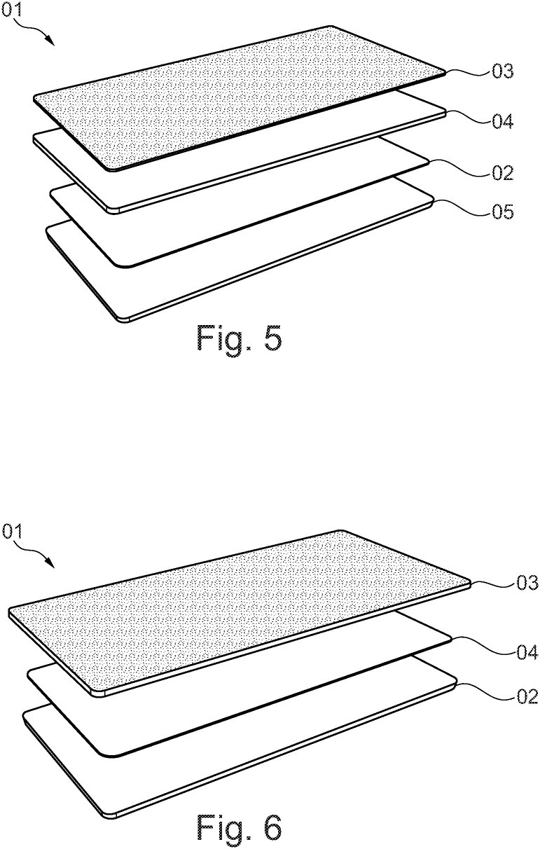

FIG. 5 is an exploded perspective view of a first exemplary embodiment of a multilayer design of a splint device 01.

FIG. 6 is an exploded perspective view of a second exemplary embodiment of a multilayer design of a splint device 01.

A splint device 01 that is shown fully or in part in FIG. 1, FIG. 2, FIG. 3, FIG. 4, FIG. 5, FIG. 6 and may be used for splinting fractures and other injuries, comprises:
at least one thin-walled strip 02 as a stiffening component, wherein a first fiber material 03 is disposed on at least one side of the thin-walled strip.
at least one strap 07 of a second fiber material 06, wherein the one and the other fiber material 03, 06 function in combination as a hook and loop fastener.

The side of the splint provided with the first fiber material 03 is also referred to as the loop side.

In order to act in combination as a hook and loop fastener, the one fiber material 03, 06 advantageously comprises flexible hooks, and the other fiber material 06, 03 advantageously comprises loops.

The splint device 01 may also be referred to as a splint.

An aluminum sheet is suitable as a stiffening component, for example. Alternatively or additionally, a thin polymer sheet may form all or part of the stiffening component.

The thin-walled strip 02 may be made of a rigid material of stable shape with respect to the tensile and compressive forces acting within the area it occupies. Advantageously, the material of the thin-walled strip 02 may be deformable in a direction perpendicular to the area it occupies, for example resiliently flexible or of plastic ductility.

Particularly preferably, the thin-walled strip 02 is made of an aluminum sheet.

The splint device 01 may be formed in one piece.

The one or more straps 07 are therefore connected to the thin-walled strip 02 comprising the first fiber material 03. The connection may be inseparable, whereby a separation of the straps 07 and the thin-walled strip 02 comprising the first fiber material 03 is only possible by mechanical destruction, for example, cutting with scissors, for instance of the straps 07 at the point of connection to the thin-walled strip 02 comprising the first fiber material 03. Alternatively, the connection may be releasable, whereby reestablishing the connection is possible. For example, the one or more straps 07 may be connected to the thin-walled strip 02 comprising the first fiber material 03 by means of the hook and loop action of the second fiber material 06.

The splint device 01 is preferably multi-layered, as shown in FIG. 5 and FIG. 6. Accordingly, the splint device 01 comprises at least the thin-walled strip 02, for example, as a core, and a top covering layer comprising the first fiber material 03.

The first fiber material 03 may form, over its entire area, a top and/or bottom face 08 of the splint device. Alternatively, it may occupy a section of a top and/or bottom face 08 of the splint device 01, as shown in FIG. 1, FIG. 2, FIG. 3.

The first fiber material 03 may, for example, form or comprise all or part of a topmost and/or undermost layer of the splint device formed by the top and/or bottom face 08. Alternatively or additionally, it may be formed or comprised by the topmost and/or undermost layer of the splint device 01 formed by the top and/or bottom face 08, for example.

Preferably, the second fiber material 06 forms or comprises the strap 07 or is formed or comprised as a whole or in part by the strap 07.

Advantageously, a plurality of straps 07 is disposed transversely along the longitudinal direction of the strip 02. Accordingly, a plurality of straps 07 is disposed transversely along the longitudinal direction of the strip 02.

One or more additional padding layers 04, 05 and/or protective layers may be disposed between the first fiber material 03 and the thin-walled strip 02 and/or on the side of the thin-walled strip 02 facing away from the first fiber material 03.

It can be seen that the invention may, for example, be implemented by a splint for holding a patient’s body parts in place. The splint is designed to be deformable, in order to be able to be transformed from a sheet material (FIG. 1, FIG. 2, FIG. 3) into a material adapted to the body part (FIG. 4). The splint is provided with a first padding layer 05, a thin-walled strip 02 attached thereto as a stiffening component, in particular an aluminum and/or metal layer such as an aluminum sheet, for example as a core, and a second padding layer 04 on top of the thin-walled strip 02.

The splint is characterized in that a fleece fabric, similar to the soft loop side of a hook and loop connection, is applied as the first fiber material 03 on top of the second padding 04, where the second padding 04, in addition or alternatively, comprises or is comprised by such a fabric.

Advantageously, the splint comprises a strap 07, onto which the second fiber material 06 is applied. Alternatively or additionally, the strap 07 may comprise or be comprised by such a fiber material 06.

The strap 07 may, for example, be cut into sections of suitable and/or required length.

Alternatively, the strap 07 may already be divided into a plurality of sections and/or attached to a plurality of locations on one side of the splint. The splint may be adapted to the body part with the free distal end(s) of the one or more straps 07 and at the same time attached to the body part by hooking it into the first fiber material 03.

It must be emphasized that the invention may be implemented by a system 10, shown as a whole or in part in FIG. 1, FIG. 2, FIG. 3, FIG. 4, FIG. 5, FIG. 6, comprising a splint device 01 or a splint described above that may be used for splinting fractures and other injuries and an additional hook strap 07 comprising the second fiber material 06. The splint, first in its initial form and then manually adapted to a body part of a patient that is to be supported, is attached, in its adapted shape, to the patient by means of the hook strap 07 via the mutual interaction of the loop side and the hook strap 07.

In the system 10, the splint device 01 is fixed to the patient by the mutual interaction of the loop side and the hook strap 07.

It must be emphasized that the idea on which the invention is based lies in a loop side formed by a loopy upper side of a splint device 01 also referred to as a splint. This loop side formed or comprised by a first fiber material 03 or forming or comprising a first fiber material 03 serves to attach hook strips. These are to be provided with the system formed by the product of splint device 01 and strap 07 and are to be used for attaching the splint to the limb to be stabilized. The use of external bandaging material is no longer necessary and the patient can be treated more quickly.

As a rudimentary implementation of the idea, a narrow piece of the first fiber material 03 may be glued as a loop side to a side forming a top and/or bottom face 08 of the splint, for attaching the splint, for example, to an arm by means of two straps 07 embodied as hook strips. The operating principle is clear.

The invention is not limited by the description of exemplary embodiments. Rather, the invention encompasses any novel feature and also any combination of features, which includes, in particular, any combination of features in the claims, even if this feature or this combination itself is not explicitly specified in the claims or exemplary embodiments.

The invention may be applied industrially in particular for producing medical technology articles, in particular those for medical emergency aid and/or first aid.

The invention has been described by reference to preferred embodiments. However, it will be apparent to a person skilled in the art that modifications or variations can be made in the invention without departing from the scope of the following claims.

List of Reference Numerals

| | |
|---|---|
| 01 | Splint device |
| 02 | Strip |
| 03 | First fiber material |
| 04 | Padding layer |
| 05 | Padding layer |
| 06 | Second fiber material |
| 07 | Strap |
| 08 | Top and/or bottom face |
| 10 | System |

1. A splint device, comprising:
at least one thin-walled strip as a stiffening component, wherein a first fiber material is disposed on at least one side of the thin-walled strip, and
a strap of a second fiber material, wherein the one and the other fiber material function in combination as a hook and loop fastener.

2. The splint device according to claim 1, wherein the splint device is formed in one piece.

3. The splint device according to claim 1, wherein the splint device comprises several layers, consisting of at least the thin-walled strip and a top cover layer comprising the first fiber material.

4. The splint device according to claim 1, wherein the first fiber material forms at least one of a top face and a bottom face over its entire area.

5. The splint device according to claim 1, wherein the first fiber material occupies a section of at least one of a top face and a bottom face.

6. The splint device according to claim 1, wherein the first fiber material forms or comprises a topmost and/or undermost layer of the splint device, and/or is formed or comprised by a topmost and/or undermost layer of the splint device.

7. The splint device according to claim 1, wherein the second fiber material forms or comprises the strap or is formed or comprised as a whole or in part by the strap.

8. The splint device according to claim 1, wherein a plurality of straps is disposed transversely along the longitudinal direction of the strip.

9. The splint device according to claim 1, wherein one or more additional padding layers and/or protective layers are disposed between the first fiber material and the thin-walled strip and/or on the side of the thin-walled strip facing away from the first fiber material.

10. The system comprising a splint device according to claim 1 for use as a splinting means for fractures and other injuries and an additional hook strap, the strap comprising the second fiber material and fixing the splint device to a body part of a patient to be splinted by means of the mutual interaction of the first fiber material and the strap.
